(19) United States
(12) Patent Application Publication
(10) Pub. No.: US 2010/0137701 A1
(43) Pub. Date: Jun. 3, 2010

(54) Band with at least one sensor to be affixed toconvex unit

(76) Inventor: Georgios Papastefanou, Ludwigshafen am Rhein (DE)

Correspondence Address: Georgios Papastefanou, Wolfsgrubenweg 3A, Ludwigshafen am Rhein, D 67069, DE

(21) Appl. No.: 12/563,120

(22) Filed: Sep. 19, 2009

(30) Foreign Application Priority Data
Sep. 19, 2008 (DE) 10 2008 042 239.8

(51) Int. Cl. A61B 5/04; A41D 20/00

(52) U.S. Cl. 600/390; 2/170

(57) ABSTRACT

A band including at least one sensor that can be attached to a unit with a convex outer surface, specifically a tubular formation similar to the extremity of a human body, featuring a sensor area with at least partly area applying the sensors on the upper plane of the unit and an attachment area. The sensor area of the band is double-layered in which one sensor carrying sensor board between the two layers is arranged so that another sensor can be attached between the outer part and the sensor. This additional sensor measures the force which is exerted by the band onto the convex device, whereas the outer layer works with the attachment part, whereas the inner layer is penetrated by at least two electrical contacts originating in the interspace of two layers, whereas a sensor is attached to the tow electrical contacts.

### TECHNICAL AREA

The invention is a band with at least one sensor that can be attached to a unit with a convex outer surface, specifically a tubular formation similar to the extremity of a human body, featuring a sensor area at least partly attached on the upper plane of the device and an attachment section.

### STATE OF TECHNOLOGY

DE 601 19 100 T2 established a sensory device that generates information concerning different physiological parameters such as heart rate, pulse rate, and so on. This device can be attached to the human body by a type of wristband so that the sensors come into contact with the skin of the subject. The device features mounted sensors in a flexible, protective case next to a fixed platform, which is connected to a processor with cabling. DE 10 2006 013 360 A1 established a way of attaching a respondent to a sensor which collects data about pulse, skin resistance, breathing, etc. and so that the average physiological reactions. The measurements can be instantaneously saved or promptly read out as the detector measures pulse or skin resistance.

The DE 103 521 88 A1 established a flexible sensor-wristband that includes multiple sensors. Such a wristband is not practical outside of a clinical field because of the lack of an aesthetically pleasing design and an unreliable application, specifically in regard to the higher investment because of cabling, which disrupts the flow of everyday life, handicapped and sensitized, which falsifies the results and arouses reservations and fears because of the cable, electricity, visibility, time commitment, which reduces the motivation to wear this type of band. For a researcher, this means a higher subject refusal rate, and the uncertainty that the sample is representative.

So that subjects accept and continuously wear the wristband, it is crucial that it is extremely comfortable, and cannot be noticed from outside of the clothes. It is important for its acceptance that the wristband is aesthetically appealing to different target groups, in other words, that there are multiple models available so that the subject can choose his/her wristband. This improves the acceptance because the subject is actively integrated into the process.

The practicality of such a wristband is also affected greatly by the type of sensors, especially those that use electrodes to transduce physiological parameters. In clinical and laboratory fields wet electrode sensors are used which are built so that the use of a gel is required regarding contact to the skin. Furthermore, the electrode sensors must be attached to the skin, typically with a large glued surface so that the interference caused by movement can be largely avoided. In the clinical field usually a suction device is used in order to apply pressure between the electrodes and the skin. So the challenge is to create a band which collects the appropriate data, reduces the interference signals so that in natural settings data can be measured simultaneously with data on the unavoidable movements. During observation, artificial signals should be identifiable, the band should be worn comfortably and have a design which is aesthetically appealing enough so that the subject acceptance rate is higher outside of the laboratory, in everyday life.

Empirical science disciplines, which deal with the study of human behaviour, reactions, and experience, for example: epidemiologic psychiatric, psychotherapeutic, and psychological research, as well as, attitudes, market, health, social, and economic research, and the general survey-method research which is used to collect individual behaviour, reactions, values, and experiences, standard survey techniques which regard the previous or current reactions or experiences of the subject. The standard technique is the survey, whether it be through direct questioning or electronic surveys, or through telephone interviews. Methods employing third-person observation are used only in small case studies, if used at all. A psychophysiological indication of inner reactions and conditions are conducted in appropriate laboratories, which the subject of study must go to.

Survey research is confronted with the problem that people in contemporary societies lead a highly individualized lifestyle in a pluralized environment. This leads to the problem that behaviour, reaction, and experiences are subject to strong daily fluctuations on the one hand, and emotional and affective components play a larger role on the other. The research has, however, shown that the validity of retrospective self-assessment regarding one's own behaviour, reaction, values, and experiences especially about inner emotional and affective reactions and conditions is limited and falsified by an imperfect memory capacity.

Considering this fact, survey research is increasingly interested and dependent on the ability to record daily fluctuating emotions and reaction indicators in real time. Conventional representative survey research in the form of market, health, social, economic, or comparable organizations does not have the time, the training, or the financial resources to meet the requirements for recording credible data regarding the continually fluctuating internal conditions.

Due to the dependence on laboratory and the necessity of observation, which demands a large time commitment on the part of the observer, measurement of psychophysiological data cannot be in cannot be considered in representative surveys.

The proposed band is an apparatus with sensors worn on the body for the recording of information regarding daily emotions and reactions to objects, people, situation, imagination, and bodily-physical experiences in real time. The apparatus uses electrical or infrared means to transfer the saved data to an external computer.

A system of subcomponents, including a microprocessor, electronic saving of recorded data, source of electricity, and sensor system can be imbedded in the wristband. The wristband is either closed peripherally, or features a detachable closure like Velcro, a snap fastener, or a belt. The sensors can encompass skin resistance, pulse rate, skin temperature, air temperature, acceleration in three Cartesian dimensions, as well as, sensors to measure the pressure with which the button was pushed.

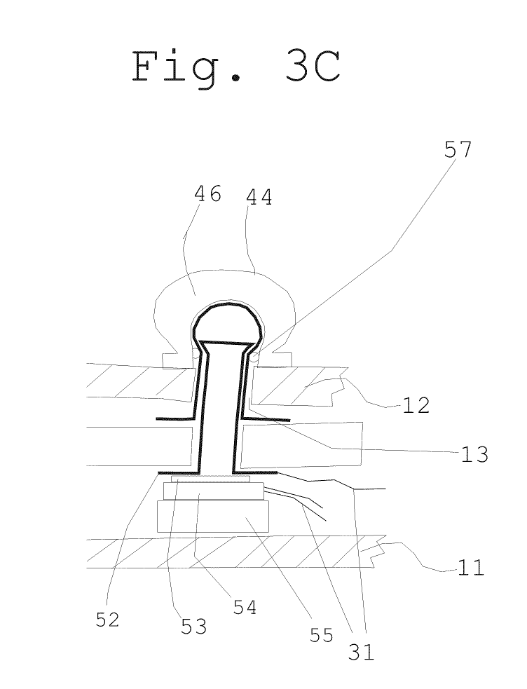

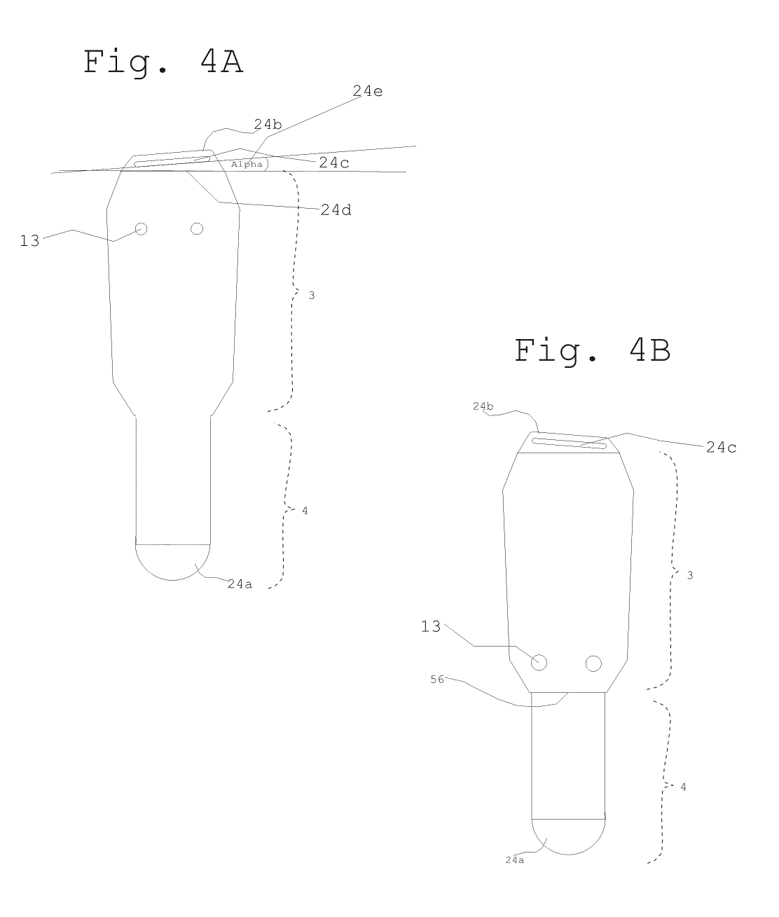

The microprocessor can encompass an Analog-Digital converter with different rates of sampling and a varying number of input ports. Subcomponents of a digital and recording system, as well as the sensors are embedded in a wristband-board material which is either woven out of elastic, electrically conductive twine, see FIGS. 4,5 or separate component boards made of rigid material like leather, metal, or a synthetic material which are preferably connected together by a electrically conductive textile element, see FIG. 6 and FIG. 7.

Aside from the elastic textile, other devices can establish the elastic bond between the separate elements. With an elastic wristband that features a separate microprocessor and sensor-architecture, the subcomponents of the microprocessor like the A/D converter, the accumulator, the operational amplifier, and the sensor inputs can be on a detached board board. The processor and sensor elements can also be constructed. The separate board elements can be connected physically and electrically to one another with a manageable, elastic textile adaptor. The separate processor and sensor elements can be attached to an elastic textile board thereby also being connected electrically.

Using elastic material to bind together separate circuit boards on which specific electronic components are distributed creates the flexible wristband.

A specific advantage of using snap fasteners as electrodes is that they are disposable such as those used in the medical field. That way, the replacement is simpler and the better quality of the transducer can be ensured. As previously mentioned, the wristband can also integrate components which convert physiologically based electrical signals into LED light, acoustic noise or vibrations. This way would be especially appealing to specific groups to communicate inner states to other people.

### DESCRIPTION OF THE INVENTION

The invention features the attributes mentioned in the first claim. Further features are explained in the sub claims.

The band integrates at least one sensor that can be attached to a unit with a convex outer surface, specifically a tubular formation similar to the extremity of a human body, featuring a sensor area on the upper plane of the device and an attachment area. The sensor area of the band is double-layered so that a sensor carrying board is arranged between the two layers, and the outer plane works with the attachment area. The inner layer is infused with at least two electric contacts, one of which must be able to attach to a sensor. For sensors with two poles, the inner layer is penetrated by at least to electrical contacts, which are located in the space between both laysers. Further the sensor is attached on at least two contacts.

The band is built both as an enclosure for the sensors and for the electronic apparatus. Advantageously, at least one of the contacts can be a projecting piece of a snap fastener made of electrically conductive material. This will achieve an electrical and mechanical connection. Unlike standard connecters that use friction to keep the plug in the jack, a snap fastener creates a stronger hold because of the use of steel springs that forcefully keep the connector in place. Furthermore, with a snap fastener, the electrical bond between the sensor and the electronic measuring equipment is guaranteed. Sensors with at least two electric contacts to the electronic measuring equipment require at least two snap fasteners.

Appropriate sensors can be chosen according to the type of measurements needed; unipolar and multipolar sensors can be used. The sensors that will be used are attached; the replacement is effortless and does not require significant amounts of money.

When the inner part is attached to the outer part with a detachable connection, the part of the device, which is turned towards the side, can easily be removed and replaced or cleaned.

The inner side can be affixed to the outer side with a detachable fastener along the edge. The inner part can also be fixed to the outer side along three sides with a permanent fastener.

The sensor board, which consists of a sensor, a fastener, and an electric piece, lies in the band; it is attached to the band solely by a snap-fastener, which also establishes the connection between the sensors and the sensor board. This offers the advantage that the inside part moves with the outside part for example during stretching.

So, the sensor board is not attached to the inside, or outside part, it is only attached by the sensor snap-fastener.

The band has two parts, the lower which touches the skin, and the upper. The two layers can be attached with Velcro and produced so that the textile encloses the sensor board with the electronic system. The layers can also be sewn or glued on three sides, so that there is an enclosure with an open side in which the sensor board can be deposited. This open side can be constructed on the shorter or the longer side of the band. For aesthetic and practical reasons, the version with the smaller side can be used more often.

Velcro is the best fastener that can be used with a textile band. Under the terms of further development, an adaptor can be attached to the end that is averted away from the fastener. That adaptor could be used for an additional supply of electricity or another device.

The sensor would preferably be attached to a sensor part so that the sensor part can maintain a flexible, electric connection with an evaluation unit. The evaluation unit and the sensor part, and the flexible fastener should be constructed as a manageable sensor boaIt would be material or a fleece, above all because an elastic material is used. If an extreminity such as a wrist or arm is turned above, below, or to the side, which happens often in everyday life, it is possible that the sensors are shoved or even lifted away from the skin which leads to increased disruption signals. If the wristband is flexible or elastic, part of the disruption can be avoided. Aside from that, an elastic band can be worn with a certain tightness, which is necessary for the vessels being put under pressure; whereas wristbands made of leather, material, or plastic would cause this pressure.

Aside from that, the material should be breathable in order to ensure that no heat accumulates under the contact sensor-skin, without the dermal tissue being pressed or the capillary wristband in order to avoid that changes of skin temperature and sweat production due to other than emotional reasons e.g. by accumulated heat instead.

It would be advantageous if the sensor board was made of textile material or a fleece and allowed the circuits to be constructed out of attached, conductive twine or filament. The sensor board should only consist of the sensor piece, the electronic piece, and the electrical connection between the two. The connection should preferably consist of one single-wired conductive cable that can be elastic or non-elastic.

This has the advantage of the wristband being pliant enough to conform to the body parts, like the wrist, so that better contact between the sensors and the skin can be achieved. Furthermore, this would ensure that the band would be thinner, and therefore less-noticeable, as well as ensuring that the apparatus could be aesthetically designed according to the subject's preferences and identity.

For a simple attachment, it is advantageous if the length of the band is measured long enough so that the band can be wrapped around once and still has a surplus length of material to attach the device at the end.

### SHORT DESCRIPTION OF THE DIAGRAMS

A model of the invention is featured in the drawings. They show:

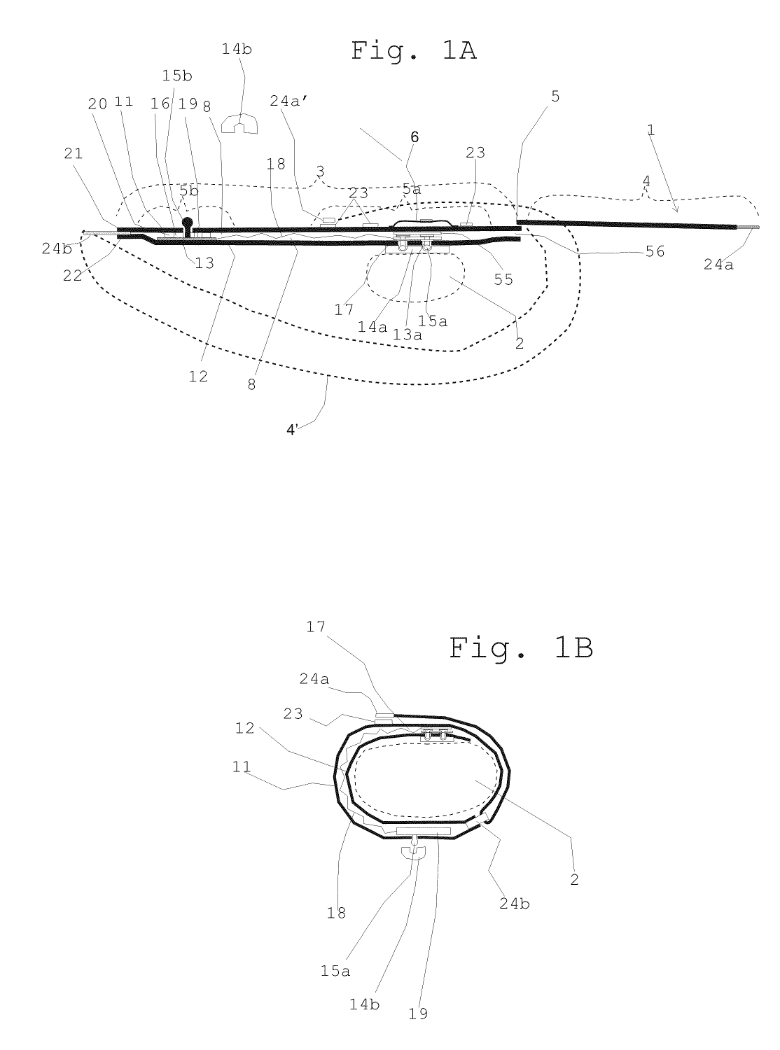

FIG. 1A: the proposed band in a stretched out condition from the side

FIG. 1B: a profile view of the proposed band attached to a body part

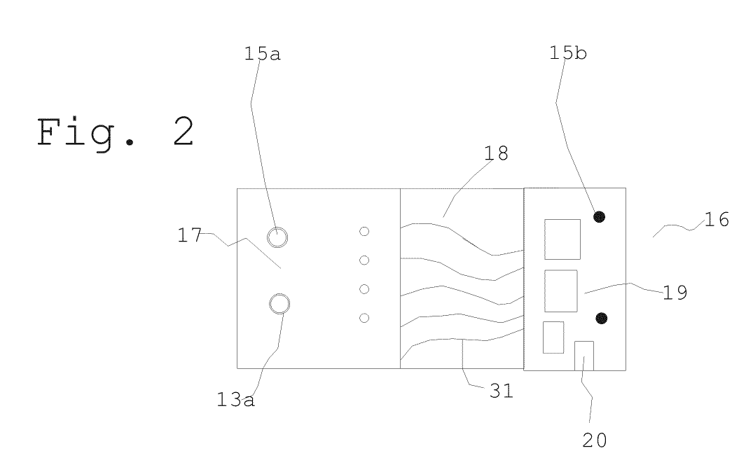

FIG. 2: a sensor board made of a sensor piece, a fastener, and electronic system for the band in FIG. 1

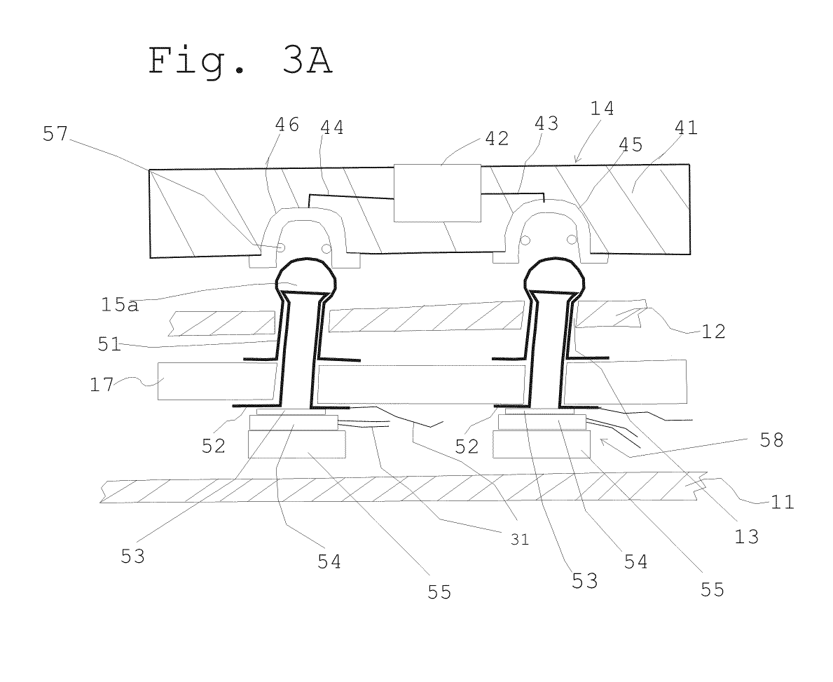

FIG. 3A: a detail for the attachment of an unplugged, bipolar sensor on the sensor piece of the sensor board

FIG. 3B: a detail for the attachment of an unplugged, unipolar sensor on the sensor piece of a sensor board

FIG. 3C: a detail for the attachment of a plugged-in, unipolar sensor on the sensor part of the sensor board

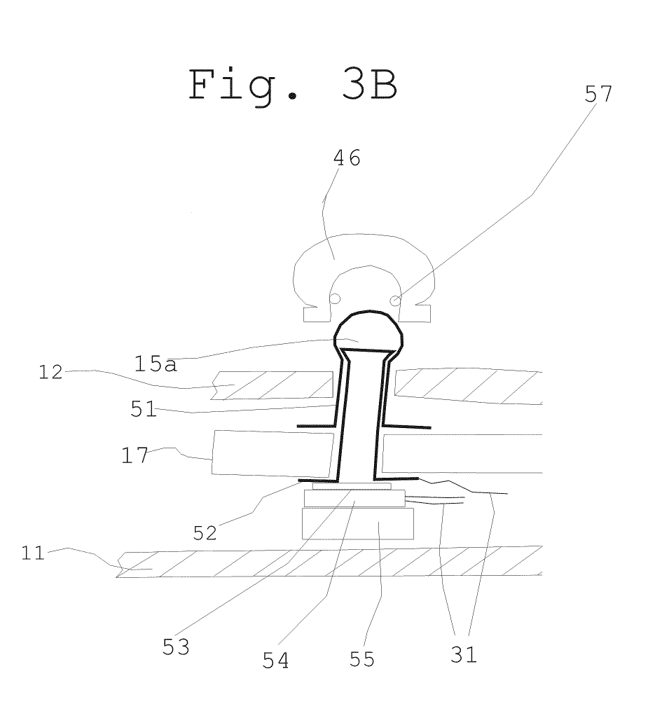

FIG. 4-7: two further models

FIG. 8A: an oversight of the top part of the proposed band

FIG. 8B: an oversight of the lower part of the proposed band

### ILLUSTRATIVE EMBODIMENT

FIG. 1A shows a flat band 1 which can be attached to a device 2 in the form of the depicted long body, especially in the form of a human extreminity.

FIG. 1B shows how the band 1 is fastened around the body 2. The band 1 features a sensor area 3 and an attachment area 4 whereby the fastener 3 as well as the fastener 4 can be wound around the length of the body. The length of the band is determined such that sensor area 3 and attachment area 4 encompass the body unit 2. Thereby can the fastener 4 with its fastening apparatus 24a, preferentially the hook part of the Velcro, run through the opening of a stiff loop on the other end of the band and back to the fastening area 5a on the top layer 11 of the band.

The material used is one that is comfortably worn. This type of band is constructed out of an elastic material that is technologically designed to attach objects to body-shaped figures, and is ideal for the arm or wrist.

According this invention the Band 1 has at least two layers in the sensor area 3 so that the outside area 11, shown as a dash, which is turned towards away from the body 2 and the inner layer 12, also shown as a dash, is turned toward the body. The inner area 12 is shorter than the outer area 11 because its function is to cover the sensor board, which is composed of an electronic part 16, a conncector 18, and the sensor part 17, so that it does not touch the body. The inner layer could cover up the whole outer area so as to avoid contact between the outer area 11 and the surface of the body 2.

With one side of the ed the inner layer 12 is connected with layer 11 by using fastening part 24b including fastening parts 21 and 21, for example like Velcro fasteners do or by glue. A removable fastener such as Velcro is intended for the attachment of the fastener part 4 with its fastening device 24a to the fastener part 23 in the connecting area 5a.

The flat, laid-out band 1 shown from the side in FIG. 1 features a depth of 10 to 100 mm on the plane of projection so as to ensure a comfortable fit for a human limb. The width of the band can be adjusted to according to specific operating conditions.

In FIG. 1, one can recognize in that the sensor board 16 (see FIG. 2) is a sensor part 17, with a sensor 14, a fastener 18, and an evaluation unit 19 which features the option of a port 20 and at least one snap fastener 15 so that the inner area of the sensor piece 17 has an opening through which the sensor, in contact with the body, 2 can be electronically and mechanically connected. As far as the evaluation unit 19 is concerned, the area 11 features an opening 13b through which the contact 15b from FIG. 1 can electrically connect at least one sensor to the evaluation unit. It would be preferable that the connection was mechanical as well.

FIG. 2 features a the sensor board 16, shown in FIG. 1 in full detail, with a reversed alignment of the sensor piece 17 and evaluation unit 19. In sensor piece 17 of the sensor board 16, the contacts 15a are intended for an electric connection to a sensor 14a (FIG. 1) which is not shown. The contacts 15a can be arranged on a flexible circuit board (not shown) and connected to the evaluation unit through a flexible fastener 18 with cables 31 or other flexible circuit paths.

The contacts 15b can be arranged on a flexible circuit board (which not showed here) and connected to an evaluation unit 19 through cables 31 or other flexible circuit paths.

With the sensor board made of textile material or fleece it is possible to connect the contact 15a and the evaluation unit 19 with a connecting part such as electronically conductive string or filament 18. In this case, one uses stitched conductive strings instead of cables 31 to keep the flexibility of the connecting part 18.

The evaluation unit 19 can come with a cable 31 through a contact point that is not shown. This end of this cable can be designed as an outlet or a terminal strip (not shown). This way, the evaluation unit 19 can be connected to an exterior device and transfer the data and programs through a port 20. This port can also be used to attach an energy supply.

In FIG. 3 the assembly of the sensor 14a in FIG. 1 is illustrated in detail. The sensor 14a consists of a base 41, in which a sensor element 42, for example a standard temperature sensor or piezo-electronic pressure sensor, is embedded so that the sensor element 42 comes into contact with its outer surface when the band is laid on the body. The sensor element 42 is electrically connected to a sensor-sided jack 45, 46 through the circuits 43 and 44. The jack can be designed to be the receiving part of the snap fastener.

The sensor 14a and its jacks 45, 46 are depicted as being located in a distance in order to improve clarity. In the attached condition, the jacks 45, 46 as well as the contact points 15a are electrically conducive connected by the latching function of a steel spring as it is use in mechanical snap fasteners.

One or more electrodes can be used in lieu of a sensor 14a in which a fastener made of an electrically conducted metal can be designated. According the mounting of sensors 14a described above button electrodes 46/47 are attached directly to contacts 15a 15b or other contacts (see also FIGS. 3 B and 3C).

In order to measure the intensity of the contact between the sensor and skin as well as the signals that lead the sensor to the evaluation unit at the same time as a parallel connection of disturbance signals which come from the reduced contact between the sensor and skin, the sensor 14a is supplied with a device 57 which measures the pressure which is exerted by the outer surface of the band 12 when the band is attached to the body part 2.

This apparatus measures the pressure that the outer band 12 exerts on the sensor 14a when it presses on the skin, mediated by the force of part 52 onto the outer part of contact 15, which is mediated to snap fastener female jack 46 and thereby mediated directly to the electrode or via the sensor board 41 onto the sensor element 42.

To measure the contact pressure which is exerted on the outside of the band 12, a manageable force resistive sensor element or piezo-electronical film sensor element 54 is put on a stiff attachment element 53 which establishes a direct connection between force sensor element 54 and inner part of sensor contact 52. Between the pressure sensor element 54 and the outer area of the band 2, a stiff element is embedded which passes on the contact pressure exerted on the pressure sensor element from the outer area 2. This stiff element 55 can be, for example, a flat battery which prepares the voltage. The pressure sensor element is connected to the evaluation unit through electric circuits or cables 31 in the attachment piece 18.

An aspect which is important to understanding the invention is that the contact 15a and 15b is plugged in as a wiring pin 15 through the hole 13a in the inner part 12 through which the sensor board 16 with the sensor piece 17 is positioned, and the sensor 14a is plugged into this contact so that the sensor board 16 is attached in the band.

With the usage of a snap fastener, contact 15a is designed in two parts, specifically with an outer part 51 and an inner part 52, which are plugged into each other and their foot-shaped, stretched-out ends of the sensor part 17 of the sensor board 16 are arranged in between. The inner part 52 is connected electronically with the cable 31, so that the sensor part 17 can be attached to a flexible circuit board.

Apart from the removable sensor 14a on the sensor part 17 of the sensor board 16 in FIG. 3A, one can also observe that the position of the sensor board 16 is determined solely by the contacts 15a, which penetrate the inner part 12.

If the inner part 12 was removed from the outer part 11 of the band 1, so that the means of attachment 21, 22 were detached, or the sensor 14a is removed, the band can be disassembled into separate pieces. For example, in the event that the outer part 11 and the inner part 12 are permanently connected on three sides, but the fourth side 56 remains detached, the sensor board, which consists of a sensor piece 17, an attachment device 18, and an evaluation unit piece 19 can be removed from the band.

These separate pieces would be the outer part 11, the inner part 12, the sensor board 16, and the sensor 14a. In the case of a permanent attachment on three sides between the inner part 12 and the outer part 11, the separate pieces would be the band 1, the sensor board 16, send the sensor 14a. If a sensor 14a is plugged into the contact 15b on the evaluation unit 16, it must be removed the same way so that the band can be disassembled into separate pieces, including the inner part 2, the outer part 11, as well as the sensor 14a and 14b and the sensor board.

Removing sensors 14a and 14b from the sensor board 16 can also be sufficient to remove the sensor board 16 laterally out of the band through the plane of projection. However, one must be sure that the contacts 15a and 15b can be removed through the hole 13 in the inner part 12 in order to avoid damage. The outer part 11 can also be integrally connected to the inner part 12 through the averted end of the attachment piece 4, however; in this case, the whole band must always be replaced or cleaned. On the side turned towards the body 2 on the outer part 11, a battery compartment can be installed so that the sensor board's power source is provided. The band is especially designed for attachment to the wrist.

External devices which can be held in the hand, for example, a printer or a keypad for individual input of the subjects' reactions and data can be attached through the port 20. Environmental sensors can be attached using the same method in which the snap fasteners on the underneath of the band employ for attaching different sensors while establishing an electronic connection. These environmental sensors could measure air temperature, light, temperature fluctuations, sonar distance sensors etc.

Output devices for signals such as LED, mini speakers, or colour-active film can be attached to the top surface of the wristband through at least one contact 15b and can, for example, also be simultaneously connected electrically using snap fasteners.

It is the same attachment principle, except that not only sensors, but also output devices can be attached to the top part. Output devices such as a mini vibration motor can also be attached to the bottom part. In this case, the outer part 11 features openings, which can be infused with contacts 15b. These contacts 15b will have sensors 46 or 41 or other data entry devices, like a miniature flat keypad, which can be attached with snap fasteners and connected electrically. Open contacts, which have no sensors attached, can be covered with dummies 14b.

In order to attach the band 1 on a convex body part 2, the attachment device 4 with the attachment element 24a is lead through the eye 24c of the tab 24b. The eye 24c is thereby not attached parallel to the end of the band 24d, instead, it is running acuate to longitudinal alignment of the band's end 24d, given a specific angle alpha 24e between middle axis of ey 24c and longitudinal alignment of band's end 24d. The size of this angle 24e is greater zero and less 45 degrees.

1. A Band with at least one sensor (14a respectively 46) to be attached to a unit (2) with a convex outer surface, especially in a tubular formation like the extremity of a human body, featuring a sensor area (3) with an at least partly attached area of the senor (14a) on the top part of the unit and an attachment area (4), shown as the band (1) in the sensor area (3) is made of double layers (11,12), whereas a sensor board (16) with sensors is arranged between the two layers (11,12), whereas the outer layer (11) works with attachment area (5a) and the inner layer (12) is penetrated by at least two contact pins (15a and 15b) which come from space 8 the between layers 11 and 12, further sensor (14a) which is oriented towards part 2 is to be attached to the at least two contact pins (15a respectively 15b) which penetrate inner layer (12).

2. A band as described in claim 1, wherein said band features both contacts on the protruding part of the snap fastener, and are made of electrically conductive material.

3. A band as described in claim 1 or 2, wherein said band's inner section (12) is connected to the exterior section (11) with a detachable attachment (21, 22).

4. A band as described in claim 1 or 2, wherein said band's inner part is permanently connected on three sides with the outer area (11), inner area (12), and outer area (11), but is not attached to the fourth side (56).

5. A band as described in claim 4, wherein said band's inner section's edge (12) is connected to the outside section with a detachable fastener (21, 22).

6. A band as described in claims 3 and 5, wherein said band uses a Velcro as a means of attachment.

7. A band as described in claims 1 to 6, wherein said band's end (24d) is attached to tab (24b), which is attached to the band's end in an acuate angle alpha (24e) relative to longitudinal axis of eye (24c).

8. A band as described in claims between 1 and 7, wherein said band has attached a bin (6) to the exterior side of the outer part (11) at the side which is averted from the fixation part (4).

9. A band as described in claims between 1 and 7, wherein said that the sensor (14a) is attached on the sensor board (17), so that sensor board (17) is connected electrically to the evaluation board (19) by a flexible and elastic connection (18, 31), wherein the sensor part (17), the elastic connection (18, 31) and the evaluation electronic part are connected and can be handled as one sensor board (16).

10. A band as described in claims 1 and 9, wherein said band is made of an elastic, textile material, especially the interior layer (12).

11. A band as described in claim 8 or 9, wherein said band's sensor board is made of an elastic textile material or fleece and that the circuit path is made of electrically charged twine or filament.

12. A band as described in the claims one through 9, wherein said band ‘s length is sufficient so that the strap 2 can reach fully around and features a surplus length so that the strap can be attached at the end.

13. Elastic wristband described in claims 11 and 12, wherein said wristband's separate processor and sensor elements are attached to an elastic and conductive textile board, as well as supplied with electricity by this textile board.

14. A sensor part with attached contacts (15a, 15b), wherein said sensor part features a sensor element 54 which is attached between the inner part (52) and the outer part (11) of the band that records the force which the outer part (11) exerts in the direction of the centre of the part (2).

15. A sensor piece with attached contacts (15a), (15b) described in claim 14, wherein said sensor piece features a connected stiff element (55) between the sensor element (54) and the outer part (11).

16. A sensor part with attached contacts (15a), (15b) described in the claims 14 and 15 wherein said sensor piece features a connected stiff element (55) between the sensor element (54) and the end of the inner part (52) of the contact (15a), (15b).
